(19) United States
(12) Patent Application Publication
(10) Pub. No.: US 2026/0258212 A1
(43) Pub. Date: Sep. 3, 2026

(54) Porous Particles And Method For Preparing The Same

(71) Applicant: LG Chem, Ltd., Seoul (KR)

(72) Inventors: Yeji KIM, Daejeon (KR); Minchae KIM, Daejeon (KR); Sengjun OH, Daejeon (KR); Jee KIM, Daejeon (KR); Yunseop KIM, Daejeon (KR)

(73) Assignee: LG Chem, Ltd., Seoul (KR)

(21) Appl. No.: 18/880,634

(22) PCT Filed: Dec. 21, 2023
(86) PCT No.: PCT/KR2023/021323

§ 371 Date: Jan. 2, 2025

(30) Foreign Application Priority Data
Feb. 1, 2023 (KR) 10-2023-0013569
May 11, 2023 (KR) 10-2023-0061331

(51) Int. Cl. C08J 9/00; C08J 9/14; C08L 29/04

(52) U.S. Cl. CPC C08J 9/0061; C08J 9/142; C08L 29/04; C08J 2203/12; C08J 2329/04; C08J 2433/14; C08L 2203/14; C08L 2312/00

(57) ABSTRACT

A method of preparing porous particles by mixing a continuous phase composition and a dispersed phase composition, the dispersed phase composition having a polymerizable monomer, a crosslinking agent, and a porogen, and a method for preparing the same and subjecting the mixture to a suspension polymerization reaction. Porous particles prepared by the method are suitable for protein purification, and affinity chromatography.

### CROSS-REFERENCE TO RELATED APPLICATIONS

This application is a 371 National Stage entry of PCT/KR2023/021323 filed on Dec. 21, 2023, which claims the benefit of Korean Patent Application No. 10-2023-0013569 filed on Feb. 1, 2023 and Korean Patent Application No. 10-2023-0061331 filed on May 11, 2023 in the Republic of Korea, the disclosures of which are incorporated by reference herein in their entirety.

### TECHNICAL FIELD

The present application relates to porous particles and a method for preparing the same. More specifically, the present application relates to porous particles for affinity chromatography and a method for preparing the same.

### BACKGROUND

As the field of biopharmaceuticals and regenerative medicine expands, the demand for systems that can efficiently separate and purify cells, tissues, microorganisms, and the like is increasing.

In conventional affinity chromatography techniques, purification resins made from hydrophilic agarose have been mainly used, but such resins are swollen by a buffer solution during the purification process and thus cause reduction in strength, which results in a deterioration of the purification speed and purification efficiency. Attempts have therefore been made to prepare synthetic polymers with improved strength.

When using purification resins, non-specific binding of impurities occurs mainly through hydrophobic interaction. The adsorbed impurities may foul the purified resin particles and have adverse effects on the activity of Protein A ligand immobilized onto the resin and the binding of the target material. Further, if impurities elute together with the purification target material, the purity of the final product decreases, and if impurities are administered to patients, they may trigger an immune response.

Therefore, there is a need to develop a technique regarding particles that can reduce non-specific adsorption, and a method for preparing the same.

### SUMMARY

### Technical Problem

It is an object of the present application to provide porous particles having an open pore system.

It is another object of the present application to provide porous particles which is reduced in non-specific adsorption.

It is another object of the present application to provide particles (e.g., column resin) for protein purification.

It is yet another object of the present application to provide porous particles suitable for affinity chromatography.

It is a further object of the present application to provide a method for preparing porous particles as described above.

The above and other objects of the present application can be all solved by the present application which is described in detail below.

### Technical Solution

According to an aspect of the present application, porous particles and a method for preparing the same are provided. The particles can be used as particles for protein purification (column resin), specifically for affinity chromatography. Herein, “porous particles” may mean particles having pores in each inside and/or on each surface of the particles. More specifically, the porous particles may mean particles having an open pore system.

Particles for protein purification (column resin) are exposed to solutions containing antibody proteins and other cell culture-derived impurities. Therefore, a situation occurs in which impurities are non-specifically adsorbed on the particle surface. In order to ensure binding between particles and target materials and increase the purity of the final product, non-specific adsorption of impurities must be lowered. The inventors of the present application have experimentally confirmed that the preparation method described below and the particles prepared thereby can reduce such a non-specific adsorption, and completed the invention of the present application.

Specifically, in order for the porous particles to have sufficient purification capacity in affinity chromatography, the target material (e.g., IgG) must be able to bind to the protein A ligand as much as possible. In order to provide such binding as much as possible, it is necessary to immobilize a large amount of the ligand to the particles because the specific surface area of the particles is high. In order to increase the specific surface area, the pores of the particles must be sufficiently large, and the number of pores (or porosity) must also be ensured at a high level. This is because, if the pores of the particles are small, the ligand may not be able to move into the inside of the particles when immobilizing the ligand, and the target material may not be able to move in the inside when purifying the target material. In addition, in order for smooth purification to proceed, the shape of the particles needs to be uniform.

Further, during the purification process, adsorption between impurity proteins and purified particles mainly occurs through hydrophobic bonds between impurity proteins and purified particles, so that as the particles have more hydrophilic groups, the hydrophobic bonds (non-specific adsorption) may be reduced.

In consideration of such points, the inventors of the present application conducted extensive research on the materials used for preparing porous particles, their content, reaction conditions, surface characteristics of the particles, and the like, and completed the disclosure of the present application.

Now, the disclosure of this application will be described in more detail.

In one aspect related to the present application, this application relates to a method for preparing porous particles. The porous particles prepared according to the method of the present application may have characteristics suitable for protein purification purposes, specifically affinity chromatography.

Specifically, the method comprises mixing a dispersed phase composition and a continuous phase composition in a reactor and then subjecting the mixture to a suspension polymerization reaction. Wherein, “dispersed phase composition” means a composition that can form a dispersed phase (or droplet) after being mixed with a continuous phase composition, and “continuous phase composition” means a composition that can form a continuous phase after being mixed with a dispersed phase composition.

In an aspect of the present application, the dispersed phase composition includes an epoxy group-containing polymerizable monomer, a crosslinking agent, and a porogen. In an aspect of the present application, the dispersed phase composition may further include an initiator in addition to the epoxy group-containing polymerizable monomer, the crosslinking agent, and the porogen. In another aspect, the dispersed phase composition may be a mixture of the above-mentioned components.

By suspension polymerization of the dispersed phase composition, a suspension polymerization product containing a unit derived from the polymerizable monomer and the crosslinking agent can be obtained. More specifically, a suspension polymerization product which includes a unit derived from the polymerizable monomer and the crosslinking agent, or is a reaction product between a polymerizable monomer (progressed by an initiator) and the crosslinking agent, can be obtained by the suspension polymerization. The suspension polymerization product may be particles, or a particle group including the same, and each individual particle may have particle characteristics described below.

In one exemplary aspect, the polymerizable monomer may be a monomer containing an unsaturated bond between carbons and an epoxy group (or glycidyl group). The type of such polymerizable monomer is not particularly limited, but may be, for example, a (meth)acrylate monomer having an epoxy group, and more specifically, glycidyl (meth)acrylate, 4,5-epoxybutyl (meth)acrylate, or 9,10-epoxy stearyl (meth)acrylate may be mentioned. However, the types of polymerizable monomers included in the dispersed phase composition of the present application are not specified by the monomers listed above.

The dispersed phase composition further comprises a crosslinking agent. In an aspect of the present application, a compound containing at least two double bonds between carbons (e.g., a vinyl group) that can participate in a crosslinking reaction may be used as the crosslinking agent.

The crosslinking agents that can be used according to aspects of the present application include, for example, polyfunctional (meth)acrylates, such as ethylene glycol di(meth)acrylate, trimethylolpropane tri(meth)acrylate, pentaerythritol tri(meth)acrylate, dipentaerythritol hexa(meth)acrylate, dipentaerythritol hexamethacrylate or trimethylpropane trimethacrylate. Alternatively, one or more of the crosslinking agents listed above may be used in combination.

In one exemplary aspect, the crosslinking agent compound may be one that does not contain a hydrophilic group (e.g., a hydroxy group). As a result of experimental confirmation, if the crosslinking agent contains a hydrophilic group in addition to the double bond between carbons, the reaction rate of the crosslinking agent may decrease, which may cause conditions that make it difficult to form sufficient particle pores.

The dispersed phase composition further includes porogen. Porogen does not participate in the polymerization reaction, but is a component that can form pores in the polymerize particles while escaping from the dispersed phase droplet during the suspension polymerization reaction.

In an aspect of the present application, the porogen may be a porogen having a difference in Hansen solubility parameters from the epoxy group-containing polymerizable monomer of 1.30 to 4.70, or may include such a porogen. Specifically, the difference in Hansen solubility parameters between the epoxy group-containing polymerizable monomer and the porogen may be 1.40 or more, 1.50 or more, 1.60 or more, 1.70 or more, 1.80 or more, 1.90 or more, 2.00 or more, 2.10 or more, 2.20 or more, 2.30 or more, 2.40 or more, 2.50 or more, 2.60 or more, 2.70 or more, 2.80 or more, 2.90 or more, 3.00 or more, 3.10 or more, 3.20 or more, 3.30 or more, 3.40 or more, 3.50 or more, 3.60 or more, 3.70 or more, 3.80 or more, 3.90 or more, or 4.00 or more. And, the upper limit thereof may be, for example, 4.60 or less, 4.50 or less, 4.40 or less, 4.30 or less, 4.20 or less, 4.10 or less, 4.00 or less, 3.90 or less, 3.80 or less, 3.70 or less, 3.60 or less, 3.50 or less, 3.40 or less, 3.30 or less, 3.20 or less, 3.10 or less, or 3.00 or less. As a result of experimental confirmation, if the difference in Hansen solubility parameters is satisfied, it is possible to provide particles with a porous structure that allows the target material to reach the center of the particles while having surface pores large enough to allow the target material to flow into the inside of the particles.

In one exemplary aspect, a non-aromatic material can be used as the porogen. For example, the porogen may be or include an acetate-based material (porogen). In an aspect, the porogen component may be at least one selected from the group consisting of butyl acetate, isobutyl acetate, amyl acetate, isoamyl acetate, hexyl acetate, isohexyl acetate, and heptyl acetate. When using an acetate-based porogen that satisfies the values related to the solubility parameters mentioned above, it is advantageous in ensuring that the particles have sufficient purification capacity in affinity chromatography applications. Specifically, when another type of porogen (e.g., aromatic porogen such as toluene or mesitylene) is used instead of the acetate-based porogen mentioned above, it was experimentally confirmed that the deviation regarding the degree of occurrence of cracked particles is large. That is, when particles are prepared as in the present application, the proportion of cracked particles can be greatly reduced (particles having no cracks are stably formed), particles with sufficiently large pores can be provided, which is therefore advantageous in reducing non-specific adsorption.

In an aspect of the present application, the dispersed phase composition may further include an initiator. The type of initiator is not particularly limited as long as it does not interfere with ensuring the particle characteristics required in the present application. For example, initiators such as an organic peroxide initiator or an azo initiator can be used. Specifically, compounds such as benzoyl peroxide, di-t-amyl peroxide, t-butyl peroxybenzoate, 2,5-dimethyl-2,5 di-(t-butylperoxy)hexane, 2,5-dimethyl-2,5-di-(t-butylperoxy)hexyne-3, di-cumyl peroxide, lauryl peroxide, 2,2′-azobis(2,4-dimethylvaleronitrile), and mixtures thereof can be used, but are not limited thereto.

In an aspect of the present application, the dispersed phase composition may include 50 parts by weight or more of the polymerizable monomer based on 100 parts by weight of the crosslinking agent. For example, the dispersed phase composition may include the polymerizable monomer in the amount of 60 parts by weight or more, 70 parts by weight or more, 80 parts by weight or more, 90 parts by weight or more, 100 parts by weight or more, 110 parts by weight or more, 120 parts by weight or more, 130 parts by weight or more, 140 parts by weight or more, 150 parts by weight or more, 160 parts by weight or more, 170 parts by weight or more, 180 parts by weight or more, 190 parts by weight or more, or 200 parts by weight or more based on 100 parts by weight of the crosslinking agent. The upper limit of the content of the polymerizable monomer in the dispersed phase composition may be, for example, 400 parts by weight or less, 350 parts by weight or less, 300 parts by weight or less, 250 parts by weight or less, 200 parts by weight or less, 150 parts by weight or less, or 100 parts by weight or less. Further, in the specific aspect of the present application, the polymerizable monomer may be included in an amount of 100 parts by weight or more, 150 parts by weight or more, or 200 parts by weight or more based on 100 parts by weight of the crosslinking agent. When the above-mentioned content is satisfied, an emulsion is stably formed, particle breakage during polymerization is greatly suppressed, and an appropriate level of particle strength can be ensured without making the pore size of the final particle excessively large or small. In particular, when the content of the polymerizable monomer and crosslinking agent satisfies the above range, the proportion of cracked particles can be reduced (particles having no cracks are stably formed), particles with sufficiently large pores can be provided, which is therefore advantageous in reducing non-specific adsorption.

In an aspect of the present application, the dispersed phase composition may contain the porogen in an amount of 45% by weight or more based on 100% by weight of the total content of the polymerizable monomer, the crosslinking agent, and the porogen. For example, based on the total content of 100% by weight, the content of porogen (which may be an acetate-based porogen by way of example) may be 50% by weight or more, 55% by weight or more, or 60% by weight or more. And, the upper limit of the content may be, for example, 70% by weight or less, 65% by weight or less, 60% by weight or less, or 55% by weight or less. If the above-mentioned content is satisfied, it is advantageous in stably forming an emulsion and ensuring particle characteristics suitable for the intended use (e.g., characteristics of less particle breakage and high spheroidization rate). In particular, when the content of polymerizable monomer and crosslinking agent satisfies the above range, the proportion of cracked particles can be reduced (particles having no cracks are stably formed), and particles with sufficiently large pores can be provided, which is therefore advantageous in reducing non-specific adsorption.

The content of the initiator is not particularly limited. The initiator may be used in an appropriate amount at a level that does not interfere with ensuring the desired particle characteristics. For example, the dispersed phase composition may contain the initiator in an amount of 0.1 parts by weight or more, specifically 0.2 parts by weight or more, 0.3 parts by weight or more, 0.4 parts by weight or more, 0.5 parts by weight or more, 0.6 parts by weight or more, 0.7 parts by weight or more, 0.8 parts by weight or more, 0.9 parts by weight or more, or 1.0 parts by weight or more with respect to 100 parts by weight of the total weight of the polymerizable monomer and the crosslinking agent. And, the upper limit of the content of the initiator may be, for example, 5.0 parts by weight or less, specifically, 4.5 parts by weight or less, 4.0 parts by weight or less, 3.5 parts by weight or less, 3.0 parts by weight or less, 2.5 parts by weight or less, 2.0 parts by weight or less, 1.5 parts by weight or less, or 1.0 parts by weight or less.

In one exemplary aspect, the continuous phase composition (A) may include water and a polymeric surfactant.

Although not particularly limited, water may be distilled water or deionized water (DIW).

In one exemplary aspect, the continuous phase composition may be a mixture of water and a polymeric surfactant. That is, the continuous phase composition may consist of only water and a polymeric surfactant.

In aspects of the present application, the polymeric surfactant may have a weight average molecular weight (Mw) in a predetermined range and/or a hydration degree (hydrolyzed degree) in a predetermined range.

In one exemplary aspect, the weight average molecular weight of the polymeric surfactant may be 60,000 or more. Specifically, the lower limit of the weight average molecular weight may be, for example, 65,000 or more, 70,000 or more, 80,000 or more, 85,000 or more, 90,000 or more, 95,000 or more, 100,000 or more 105,000 or more 110,000 or more 115,000 or more 120,000 or more 125,000 or more 130,000 or more 135,000 or more 140,000 or more. And, the upper limit thereof may be 200,000 or less. Specifically, the upper limit of the weight average molecular weight may be, for example, 190,000 or less, 185,000 or less, 180,000 or less, 175,000 or less, 170,000 or less, 165,000 or less, 160,000 or less, 155,000 or less, 150,000 or less, 145,000 or less, 140,000 or less, 135,000 or less, 130,000 or less, 125,000 or less, 120,000 or less, 115,000 or less, or 110,000 or less. The weight average molecular weight can be measured using GPC.

In one exemplary aspect, the polymeric surfactant may have a hydration degree in the range of 80 to 99%. More specifically, the lower limit of the hydration degree may be, for example, 81% or more, 82% or more, 83% or more, 84% or more, or 85% or more, and the upper limit thereof may be, for example, 98% or less, 97% or less, 96% or less, 95% or less, 94% or less, 93% or less, 92% or less, 91% or less, or 90% or less. For example, polyvinyl alcohol can be used as a surfactant having the above hydration degree. The hydration degree can be measured using 1H-NMR.

Examples of polymeric surfactants that can be used include poly(vinyl alcohol) (PVA) or polyvinylpyrrolidone.

In one exemplary aspect, in the continuous phase composition, the concentration of the polymeric surfactant may be 1.0% or more. For example, when using a continuous phase composition which is a mixture of water and a polymeric surfactant, the content of the polymeric surfactant may be 1.0% by weight or more based on the total weight (100% by weight) of the continuous phase composition. Specifically, the lower concentration limit of the polymeric surfactant in the continuous phase composition (A) may be 1.5% by weight or more, 2.0% by weight or more, 2.5% by weight or more, 3.0% by weight or more, or 3.5% by weight or more, and the upper limit of the polymeric surfactant concentration may be less than 4.0%, specifically, for example, 3.5% or less, 3.0% or less, 2.5% or less, 2.0% or less, or 1.5% or less. When a polymeric surfactant that satisfies the above concentration is used, uniform and stable droplets can be formed when mixing the continuous phase and the powder phase, which is advantageous in obtaining the shape of the final particle intended by the present application.

In one exemplary aspect, the weight ratio of the dispersed phase composition to the continuous phase composition (weight ratio of the dispersed phase composition: weight ratio of the continuous phase composition) may be in the range of 1:2 to 1:15, or in the range of 1:4 to 1:12, or in the range of 1:6 to 1:10.

The suspension polymerization can be performed under conditions that do not interfere with ensuring particle characteristics according to the preparation method of the present application. For example, the suspension polymerization reaction may be performed at a temperature of 100° C. or less, more specifically at a temperature of 40 to 95° C. Further, during the polymerization reaction at the above temperature, stirring can be performed at a speed of 600 to 1,000 rpm.

In one exemplary aspect, the method may further include a washing step after completion of the suspension polymerization reaction. By this washing, impurities unrelated to particles which are the suspension polymerization product can be removed. The washing method is not particularly limited, and any known washing method can be used. For example, the washing may be performed by adding the suspension polymerization product to a washing solution containing alcohol (e.g., alcohol containing ethanol) and/or high-temperature (e.g., 40~60° C.) water (e.g., distilled water) followed by stirring. Although not particularly limited, this washing may be repeated, for example, two or more times.

In one exemplary aspect, the method may further include a drying step after the washing. Solvent residues and the like can be removed by drying. The drying method is not particularly limited, and any known drying method can be used. For example, the drying can be performed using an oven or under room temperature conditions. Further, although not particularly limited, the drying may be performed in a vacuum.

In one exemplary aspect, the method may further include centrifuging, washing, and drying the suspension polymerization product after completion of the suspension polymerization reaction. The details regarding the washing and drying are the same as those described above.

The suspension polymerization product, i.e., particles, prepared according to the above method including suspension polymerization, may have characteristics suitable for protein purification or separation applications.

In one exemplary aspect, the particles prepared according to the method of the present application may have a size within the range of 30 to 70 μm. Herein, the particle size means the particle size (D₅₀) measured using PSA (particle size analysis) device. And, D₅₀ means the particle size corresponding to 50% cumulative volume in the particle size distribution. Specifically, the particles prepared according to the present application may have a lower limit size of 35 μm or more, 40 μm or more, or 45 μm or more, and an upper limit size of 65 μm or less, 60 μm or less, 55 μm or less, 50 μm or less, or 45 μm or less. In the case of exceeding the above particle range, the number of particles that can be packed in the purification column decreases, the content of the ligand attached to the particle surface decreases, and ultimately the purification efficiency may decrease. In addition, when the particle size is smaller than the above particle size, the density of the particles (packed beads) packed in the purification column becomes excessively large, making it difficult for the solution to be purified to escape, which may be therefore lowered in the purification efficiency.

In one exemplary aspect, the method of the present application can provide a particle group with a low percentage of cracked particles. For example, the proportion of the number of cracked particles may be 20% or less, 15% or less, 10% or less, or 5% or less of the total particles prepared. That is, according to the present application, particles having a stable shape are provided. If the proportion of cracked particles is high, it can be seen that the preparation efficiency is low. Furthermore, when particles are used for protein purification, the purification efficiency may decrease as cracked particles clog the channels through which the solution flows, and the product purity may decrease as cracked particles escape through the column discharge port.

In one exemplary aspect, the particles may exhibit a high spheroidization rate. For example, the spheroidization rate calculated as the ratio of the particle size (D₅₀) to the length (L) of the particles having the longest dimension length (L) among the prepared total particles may be 80% or more, 85% or more, or 90% or more. That is, according to the present application, uniform particles with a high degree of spheroidization are provided. If the spheroidization rate is low, the purification efficiency may be lowered because the particles are not sufficiently packed in the column.

In one exemplary aspect, the particles may have a pore size of 30 nm or more when the pore size is measured using a mercury intrusion method. The mercury intrusion method is a measurement method that converts the pore size through the amount of mercury introduced by pressure from the surface to the inside of the particles. According to aspects of the present application, the measured data related to the mercury intrusion method may be represented by a histogram, and the size of the particle pores can be calculated from the measurement of the total intrusion volume at the maximum mercury injection pressure and the ratio of the total pore area (wherein, the size of the particle pores can be considered an average size, and the average can mean an arithmetic mean). If the pores of the particle are too small, only impurities with a small size relative to the target protein will flow into and be adsorbed inside the particle, which may lead to a reduction in purification efficiency due to an increase of non-specific adsorption. For example, the pore size of the particles may be 35 nm or more, 40 nm or more, 45 nm or more, 50 nm or more, 55 nm or more, or 60 nm or more. Further, the upper limit of the average pore size of the particles may be 500 nm or less. When the average pores of the particles are too large, the retention time of impurities in the interparticle voids smaller than the particle pores may increase, and the purification efficiency may decrease due to an increase of non-specific adsorption. Further, due to a decrease in the specific surface area inside the particles, the content of attached ligand may decrease, which may be therefore lowered in the purification efficiency. For example, the pore size of the particles may be 400 nm or less, 300 nm or less, 200 nm or less, or 100 nm or less.

In one exemplary aspect, the average surface pore size of the particles may be in the range of 110 to 240 nm. In an aspect of the present application, the average surface pore size is related to the degree of openness of the particle surface, and may mean an (arithmetic) average value calculated after measuring 20 or more pore sizes (wherein, the pore size means the length of the largest dimension in the pore shape) on an SEM image of the particle surface. It is not possible to ascertain the degree of openness of the particle surface only by the pore size of the particle determined by the mercury intrusion method. Considering the flow of the target material or fluid through the particles, the specific surface area of the particles, the mechanical strength of the particles, and the like, the average surface pore sizer may be, for example, 120 nm or more, 130 nm or more, 140 nm or more, 150 nm or more, 160 nm or more, 170 nm or more, 180 nm or more, 190 nm or more, or 200 nm or more, and the upper limit thereof may be, for example, 230 nm or less, 220 nm or less, 210 nm or less, 200 nm or less, 190 nm or less, 180 nm or less, 170 nm or less, 160 nm or less, or 140 nm or less.

In one exemplary aspect, the porosity of the particles may be in the range of 15 to 40%. Specifically, the porosity of the particles may be, for example, 16% or more, 17% or more, 18% or more, 19% or more, 20% or more, 21% or more, 22% or more, 23% or more, 24% or more, 25% or more, and 39% or less, 38% or less, 37% or less, 36% or less, 35% or less, 34% or less, 33% or less, 32% or less, 31% or less, or 30% or less. If the porosity is smaller than the above-mentioned range, it is difficult to expect high purification efficiency because the specific surface area of the particles is low. If the porosity exceeds the above-mentioned range, the mechanical strength may deteriorate.

In an aspect of the present application, the method further comprises a predetermined surface treatment step for the suspension polymerization product. Specifically, the method further includes a step of making the surface of the suspension polymerization product hydrophilic through deactivation of epoxy groups.

The particles obtained by the above-mentioned suspension polymerization have an epoxy group derived from an epoxy group-containing polymerizable monomer. In the present application, an epoxy deactivator (e.g., a compound having an epoxy reactive group and a hydrophilic group different therefrom) is reacted with the particles formed by the suspension polymerization to deactivate epoxy groups on the surface of the particles and introduce a hydrophilic group to the surface of the particles. By introduction of the hydrophilic group through epoxy group deactivation, non-specific adsorption caused by hydrophobic bonds between impurity proteins and purified particles can be reduced.

As mentioned above, in an aspect of the present application, at least 50 parts by weight of the epoxy group-containing polymerizable monomer is used relative to 100 parts by weight of the crosslinking agent, so that the particles, which are the suspension polymerization product, have a sufficient amount of epoxy groups on their surfaces. Since a sufficient amount of epoxy groups on the particle surface is ensured, the particle surface can be sufficiently hydrophilized due to epoxy deactivation when the epoxy deactivator and the particles react with each other.

The hydrophilization may be performed by reacting an epoxy deactivator having an epoxy reactive group and a hydrophilic group different therefrom with the suspension polymerization product having the epoxy group. For example, the above-mentioned epoxy group deactivation can be achieved by injecting the prepared particles into an epoxy deactivator or a solution containing it. The solvent used at this time is not particularly limited. Further, the reaction for making the surface hydrophilic can be performed, for example, within the range of 30 to 50° C. for 10 to 30 hours.

In one exemplary aspect, the epoxy deactivator may be a compound having an epoxy reactive group and a hydrophilic group different therefrom. The epoxy reactive group may be, for example, a thiol group or an amine group, and the hydrophilic group different from the epoxy reactive group may be, for example, a hydroxy group.

For example, the deactivator may include at least one selected from 1-thioglycerol, ethanolamine, and 2-mercaptoethanol. For example, when 1-thioglycerol is used as a deactivator, deactivation of the epoxy group occurs by an epoxy-thiol reaction, and when ethanolamine is used as a deactivator, deactivation of the epoxy group occurs by an epoxy-amine reaction, wherein the former case is more advantageous in terms of reaction efficiency. Therefore, it may be preferable to use an epoxy deactivator, such as 1-thioglycerol, which contains a thiol group as an epoxy reactive group and a hydroxy group as a hydrophilic group different from the epoxy reactive group.

In an aspect of the present application, the method comprising: mixing a continuous phase composition and a dispersed phase composition and then subjecting the mixture to a suspension polymerization reaction to prepare a suspension polymerization product including a unit derived from the polymerizable monomer and the crosslinking agent; and making the surface of the suspension polymerization product hydrophilic through epoxy group deactivation can provide porous particles that exhibit low non-specific adsorption rates. For example, the method can provide the porous particles having a non-specific adsorption rate to BSA (Bovine serum albumin) of 1.5% or less. Specifically, the particles prepared according to the above method can satisfy a non-specific adsorption rate for BSA (Bovine serum albumin) of 1.0% or less, more specifically, 0.5% or less, 0.45% or less, 0.4% or less, 0.35% or less, 0.3% or less, 0.25% or less, 0.2% or less, 0.15% or less, 0.1% or less, 0.05% or less, or 0.01% or less. The non-specific adsorption rate is measured for a mixture containing porous particles, BSA, and water, and is calculated as the ratio of the content (weight) of BSA adsorbed on the porous particles to the content (weight) of the total BSA added to the mixture. According to an aspect of the present application, the porous particles and the BSA in the mixture for measuring the non-specific adsorption rate may be included in a weight ratio of 1 to 3 porous particles: 1 BSA.

In one exemplary aspect, the above method can provide particles that satisfy a predetermined hydrophilic group content. Specifically, the method can provide particles that satisfy a hydrophilic group content (μmol/g) per particle unit weight of 500 μmol/g or more, specifically, 1000 μmol/g or more. Wherein, the content of hydrophilic groups per particle unit weight (μmol/g) can be calculated by multiplying the epoxy group content (μmol/g) of the suspension polymerization product, which is obtained by suspension polymerization before the surface hydrophilization treatment, by the number of hydrophilic groups in the epoxy deactivator. In addition, the epoxy group content (μmol/g) of the suspension polymerization product obtained by suspension polymerization can be calculated by titrating particles of a predetermined weight using 0.1M NaOH to calculate the epoxy group content (μmol), and dividing it by the weight of the particles. More specifically, it can be calculated according to the method described in the experiments described below. For example, the hydrophilic group (e.g., hydroxyl group) content (μmol/g) of the particles provided according to the above method may be 1500 μmol/g or more, 2000 μmol/g or more, 2500 μmol/g or more, 3000 μmol/g or more, 3500 μmol/g or more, 4000 μmol/g or more, or 4500 μmol/g or more. And, the upper limit of the hydrophilic group content (μmol/g) of the particles may be, for example, 5000 μmol/g or less, specifically 4500 μmol/g or less, 4000 μmol/g or less, 3500 μmol/g or less, or 3000 μmol/g or less.

By treatment with an epoxy deactivator as described above, particles having a hydrophilic group within the above range can provide excellent purification efficiency. Specifically, in the purification process, adsorption between impurity proteins and purified particles occurs mainly through hydrophobic bonds between the impurity proteins and purified particles. Therefore, as the particles have more hydrophilic groups at an appropriate level, the hydrophobic bonds (non-specific adsorption) can be reduced. This tendency is also confirmed in Examples described below.

In one exemplary aspect, as a result of the epoxy deactivation and hydrophilic treatment as described above, the method can provide porous particles in which the content of epoxy groups on the surface is adjusted to a predetermined level or less. Specifically, the epoxy group content (μmol) per unit weight (g) of the epoxy-deactivated particles may be 50 μmol/g or less. This epoxy group content (μmol/g) can be calculated by titrating particles of a predetermined weight using 0.1M NaOH to calculate the epoxy group content (μmol) and dividing it by the weight of the particles. More specifically, it can be calculated according to the method described in the experiments described below. Herein, the epoxy group content (μmol/g) can be calculated by titrating particles of a predetermined weight using 0.1M NaOH to calculate the epoxy group content (μmol) and dividing it by the weight of the particles. More specifically, it can be calculated according to the method described in the experiments described later. Specifically, the epoxy content of the epoxy deactivated particles may be, for example, 45 μmol/g or less, 40 μmol/g or less, 35 μmol/g or less, 30 μmol/g or less, 25 μmol/g or less, 20 μmol/g or less, 15 μmol/g or less, 10 μmol/g or less, or 5.0 μmol/g or less. The epoxy group of the particles of the present application is derived from a polymerizable material for particle formation, that is, a polymerizable monomer having an epoxy group. However, assuming that the particles are actually used for purifying the target material, if too many epoxy groups remain on the particle surface, these epoxy groups may react or bind with other materials in addition to the target material (e.g. impurities), which may therefore reduce the purification efficiency. Taking this into consideration, particles with deactivated epoxy groups are used in the present application. In addition, the lower limit of the epoxy group content (μmol) per unit weight of epoxy deactivated particles may be 1 μmol/g or more, 5 μmol/g or more, 10 μmol/g or more, 15 μmol/g or more, 20 μmol/g or more, 25 μmol/g or more, 30 μmol/g or more, 35 μmol/g or more, or 40 μmol/g or more. For reference, if the epoxy group content on the surface of the particles before deactivation is too small, the surface hydrophilization treatment by epoxy deactivation may not be sufficiently performed. Accordingly, when the particles are used as an actual column resin, it may not be able to bind sufficiently to the material that must be bound to the particles (e.g. rProtein A), and thus, it is necessary to use a sufficient amount of the epoxy group-containing polymerizable monomer as described above.

Although not particularly limited, after the epoxy deactivation and hydrophilization treatments, the particles can be washed and/or dried. The washing and drying can be performed in the same manner as described above.

In one aspect related to the present application, this application relates to porous particles. The porous particles can be prepared by the method mentioned above and, for example, can be used as a column resin for protein purification or separation.

In one aspect of the present application, the particles contain a polymerization unit derived from the polymerizable monomer having an epoxy group and the crosslinking agent. The types and characteristics of the polymerizable monomer having an epoxy group and the crosslinking agent are the same as described above.

In one exemplary aspect, the polymerizable monomer may be a monomer containing a carbon-carbon unsaturated bond and an epoxy group (or glycidyl group). The type of such polymerizable monomers is not particularly limited. For example, the polymerizable monomer may include a (meth)acrylate monomer having an epoxy group, more specifically, glycidyl (meth)acrylate, 4,5-epoxybutyl (meth)acrylate, or 9,10-epoxy stearyl (meth)acrylate. However, the types of polymerizable monomers contained in the dispersed phase composition of the present application as these monomers are not specified.

In one exemplary aspect, a polyfunctional (meth)acrylate can be used as a crosslinking agent. Specifically, the polyfunctional (meth)acrylates, such as ethylene glycol di(meth)acrylate, trimethylolpropane tri(meth)acrylate, pentaerythritol tri(meth)acrylate, dipentaerythritol hexa(meth)acrylate, dipentaerythritol hexamethacrylate or trimethylpropane trimethacrylate can be used as a crosslinking agent. Alternatively, one or more of the crosslinking agents listed above may be used in combination. However, the crosslinking agent is not limited to the materials listed above.

In addition, the particles of the present application may further include materials derived from the above-mentioned preparation method.

In one exemplary aspect, the porous particles may have a particle size (D₅₀) within the range of 30 to 70 μm. As the description thereof is the same as those set forth above, further discussions on this matter will not be provided.

In one exemplary aspect, the porous particles may have a ratio of the number of cracked particles to the total particles of 20% or less. As the description thereof is the same as those set forth above, further discussions on this matter will not be provided.

In one exemplary aspect, the porous particles can satisfy a spheroidization rate of 80% or more. As the description thereof is the same as those set forth above, further discussions on this matter will not be provided.

In one exemplary aspect, the porous particles may have a pore size of 30 nm or more. As the description thereof is the same as those set forth above, further discussions on this matter will not be provided.

In one exemplary aspect, the porous particle may have a surface pore size ranging from 110 to 240 nm. As the description thereof is the same as those set forth above, further discussions on this matter will not be provided.

In one exemplary aspect, the porous particles may have a porosity ranging from 15 to 40%. As the description thereof is the same as those set forth above, further discussions on this matter will not be provided.

In one exemplary aspect, the particles may be hydrophilically-treated, i.e., surface-treated with an epoxy deactivator having an epoxy reactive group and a hydrophilic group different from the epoxy reactive group.

Epoxy deactivation and hydrophilic treatment for particles may mean that the particles have been surface-treated with an epoxy deactivator having an epoxy reactive group and a hydrophilic group different from the epoxy reactive group. For details of such surface treatment, refer to those set forth above.

In one exemplary aspect, the porous particles may be particles that satisfy a non-specific adsorption rate for BSA (Bovine serum albumin) of 1.5% or less. As the description thereof is the same as those set forth above, further discussions on this matter will not be provided.

In one exemplary aspect, as a result of the epoxy deactivation and hydrophilic treatment as mentioned above, the porous particles can satisfy a predetermined hydrophilic group content. Specifically, the porous particle can satisfy a hydrophilic group content (μmol/g) per unit weight of the epoxy-deactivated particle of 500 μmol/g or more, specifically, 1000 μmol/g or more. As the description thereof is the same as those set forth above, further discussions on this matter will not be provided.

In one exemplary aspect, as a result of the epoxy deactivation and hydrophilic treatment as mentioned above, the porous particles may satisfy a predetermined epoxy group content. Specifically, the porous particles may have an epoxy group content (μmol) per unit weight (g) of the epoxy-deactivated particle of 50 μmol/g or less. As the description thereof is the same as those set forth above, further discussions on this matter will not be provided.

### Advantageous Effects

According to an aspect of the present application, when used for protein purification particles (column resin), particles capable of improving non-specific adsorption of impurities to the particles are provided. Accordingly, the binding capacity of the antibody protein to the particles may be sufficient, and high purity of the final product can be ensured.

### BRIEF DESCRIPTION OF THE DRAWINGS

FIGS. 1 to 7 are images of the surface of particles (base resin) prepared in Examples and Comparative Examples.

FIG. 1 is an image of the surface of the particles (base resin) of Example 1 taken using an SEM device.

FIG. 2 is an image of the surface of the particles (base resin) of Example 2 taken using an SEM device.

FIG. 3 is an image of the surface of the particles (base resin) of Example 3 taken using an SEM device.

FIG. 4 is an image of the surface of the particles (base resin) of Comparative Example 5 taken using an SEM device.

FIG. 5 is an image of the surface of the particles (base resin) of Comparative Example 6 taken using an SEM device.

FIG. 6 is an image of the surface of the particles (base resin) of Comparative Example 7 taken using an SEM device.

FIG. 7 is an images of the surface of the particles (base resin) of Comparative Example 11 taken using an SEM device.

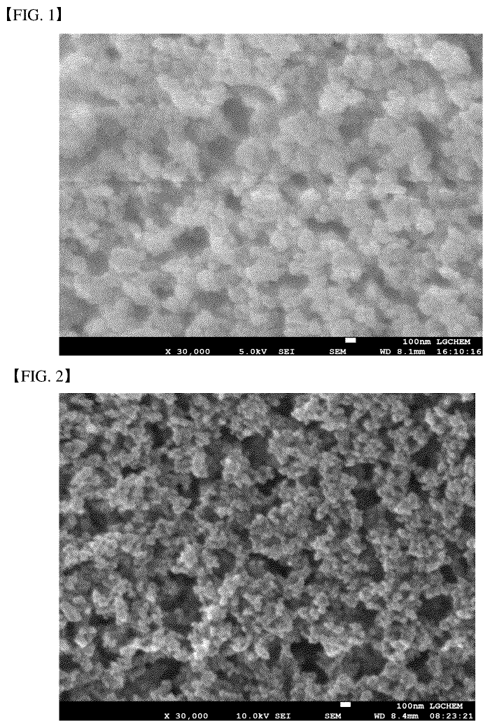

In FIGS. 1-7, the white bar represents the size of 100 nm, and the black parts observed in the particles are pores.

### DETAILED DESCRIPTION

Hereinafter, actions and effects will be described in more detail with reference to specific examples. However, this is for illustrate purposes only, and are not intended to limit the scope of the disclosure in any way.

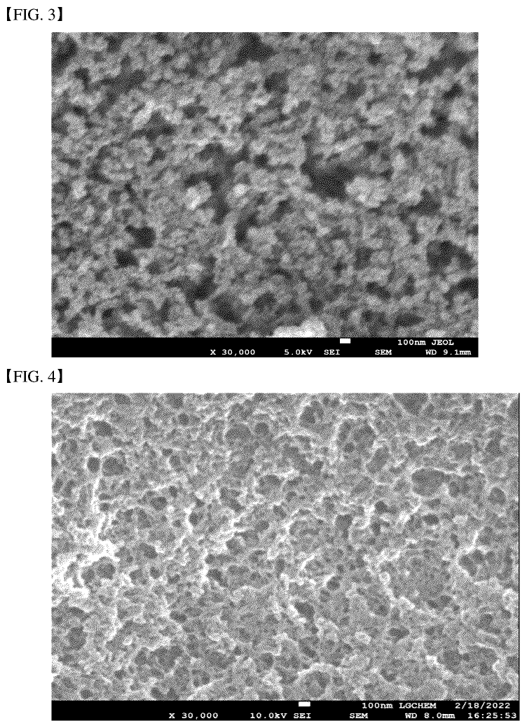

In FIGS. 1-3 of the Examples and FIGS. 4-7 of the Comparative Examples, the black parts, i.e., pores, are more visible in the images of Examples than in the images of Comparative Examples. The pores or surface pores of the particles of Examples are larger and more numerous than those of the particles of Comparative Examples. In the case of Comparative Examples (e.g. Comparative Examples 5~7), the pore size measured by MIP of the particles is at the level of 10~11 nm, and in the case of Examples (e.g., Examples 1~2), the pore size measured by MIP of the particles is consistent with the experimental results below (see Tables 5~6), which state that the pore size is at the level of 35~40 nm. Further, looking at the experimental results in Tables 5~6, it is confirmed that Examples (120~160 nm level) and Comparative Examples (level of 105 nm or less. Particularly, Comparative Example 11 is confirmed to be 45 nm) show a clear difference in average surface pore size.

### EXAMPLE AND COMPARATIVE EXAMPLE

The particle-forming components, their contents, and process conditions are as listed in Tables below, and the conditions under which each particle was prepared in Examples and Comparative Examples are as follows. If there is no specific explanation regarding temperature, the temperature at which the preparation process is performed (or each preparation step) or the temperature at which the characteristics possessed by the prepared particles are calculated or measured is room temperature (temperature without reduced or heated temperature, refers to a temperature within the range of about 15 to 30° C.).

### Preparation of Dispersed Phase

After stirring a polymerizable monomer (M), a crosslinking agent (C), and porogen (P), benzoyl peroxide (BPO), which is a thermal initiator (about 1.25 parts by weight relative to 100 parts by weight of the total mass of the polymerizable monomer and the crosslinking agent) was further added, and stirred at room temperature for about 5 minutes to prepare a dispersed phase.

In the following Examples and Comparative Examples GMA refers to glycidyl metahacrylate, EGDMA refers to ethylene glycol dimethacrylate, and TMPTMA refers to trimethylolpropane trimethacrylate

### Preparation of Continuous Phase

PVA (weight average molecular weight (Mw) of 85,000 to 124,000 and hydrolysis rate of 87 to 89%) was dissolved in distilled water at a concentration of 2 wt. % to prepare a continuous phase.

### Preparation of Particles (Base Resin) by Suspension Polymerization

720 g of the continuous phase was added to a reactor, and 90 g of the dispersed phase was added, and the mixture was stirred at about 800 rpm at room temperature until a uniform dispersion liquid was formed. Subsequently, the reactor temperature was raised, and polymerization was performed at a temperature of about 80° C., about 800 rpm, and nitrogen purging conditions for about 6 hours.

### Obtaining Particles

The polymerized particles were recovered, washed twice with distilled water at 50° C., and five times with ethanol. Subsequently, it was dried in an oven at about 80° C. to obtain particles.

TABLE 1
Preparation conditions of particles (base resin) of Examples 1~4

| | | Example 1 | Example 2 | Example 3 | Example 4 |
|---|---|---|---|---|---|
| Particle | Polymerizable | GMA | GMA | GMA | GMA |
| component | monomer (M) | | | | |
| | Crosslinking | EGDMA | EGDMA | EGDMA | EGDMA |
| | agent (C) | | (C1) + | (C1) + | |
| | | | TMPTMA(C2) | TMPTMA(C2) | |
| | Porogen(P) | Amyl acetate | Amyl acetate | Amyl acetate | N-butyl |
| | | | | | acetate |
| | Weight ratio | 2(M):1(C):3(P) | 2(M):0.5(C1):0.5(C2):3(P) | 1.5(M):0.6(C1):0.4(C2):3(P) | 2(M):1(C):3(P) |
| | Difference in | 4.34 | 4.34 | 4.34 | 3.95 |
| | solubility | | | | |
| | parameters | | | | |
| | between | | | | |
| | polymerizable | | | | |
| | monomer and | | | | |
| | porogen | | | | |

TABLE 2
Preparation conditions of particles (base resin) of Comparative Examples 1~4

| | | Comparative | Comparative | Comparative | Comparative |
|---|---|---|---|---|---|
| | | Example 1 | Example 2 | Example 3 | Example 4 |
| Particle | Polymerizable | GMA | GMA | GMA | GMA |
| component | monomer(M) | | | | |
| | Crosslinking | EGDMA | EGDMA | EGDMA(C1) + | EGDMA(C1) + |
| | agent (C) | | | TMPTMA(C2) | TMPTMA(C2) |
| | Porogen(P) | Toluene | Mesitylene | Mesitylene | Toluene |
| | Weight ratio | 1(M):3(C):3(P) | 1(M):3(C):3(P) | 1(M):1.5(C1):1.5(C2):4(P) | 1(M):1.5(C1):1.5(C2):6(P) |
| | Difference in | 8.21 | 9.73 | 9.73 | 8.21 |
| | solubility | | | | |
| | parameters | | | | |
| | between | | | | |
| | polymerizable | | | | |
| | monomer and | | | | |
| | porogen | | | | |

TABLE 3
Preparation conditions of particles (base resin) of Comparative Examples 5~8

| | | Comparative | Comparative | Comparative | Comparative |
|---|---|---|---|---|---|
| | | Example 5 | Example 6 | Example 7 | Example 8 |
| Particle | Polymerizable | GMA | GMA | GMA | GMA |
| component | monomer (M) | | | | |
| | Crosslinking | EGDMA | EGDMA | EGDMA(C1) + | EGDMA(C1) + |
| | agent (C) | | | TMPTMA(C2) | Glycerin |
| | | | | | dimethacrylate(C2) |
| | Porogen (P) | Amyl | Hexyl | Hexyl | Mesitylene |
| | | acetate | acetate | acetate | |
| | Weight ratio | 1(M):3(C):3(P) | 1(M):3(C):3(P) | 1(M):1.5(C1):1.5(C2):4(P) | 1(M):1.5(C1):1.5(C2):3(P) |
| | Difference in | 4.34 | 4.74 | 4.74 | 9.73 |
| | solubility | | | | |
| | parameters | | | | |
| | between | | | | |
| | polymerizable | | | | |
| | monomer | | | | |
| | and porogen | | | | |

TABLE 4
Preparation conditions of particles (base resin) of Comparative Examples 9~12

| | | Comparative | Comparative | Comparative | Comparative |
|---|---|---|---|---|---|
| | | Example 9 | Example 10 | Example 11 | Example 12 |
| Particle | Polymerizable | GMA | GMA | GMA | GMA |
| component | monomer(M) | | | | |
| | Crosslinking | Glycerin | EGDMA (C1) + | EGDMA | EGDMA |
| | agent (C) | dimethacrylate(C) | Glycerin | | |
| | | | dimethacrylate | | |
| | | | (C2) | | |
| | Porogen(P) | Mesitylene | Amyl acetate | Dimethyl | Glycerol |
| | | | | adipate | diacetate |
| | Weight ratio | 1(M):3(C):3(P) | 1(M):1.5(C1):1.5(C2):3(P) | 2(M):1(C):3(P) | 2(M):1(C):3(P) |
| | Difference in | 9.73 | 4.34 | 1.28 | 6.88 |
| | solubility | | | | |
| | parameters | | | | |
| | between | | | | |
| | polymerizable | | | | |
| | monomer | | | | |
| | and porogen | | | | |

### Evaluation 1: Characteristic Evaluation of Base Resin

The particles prepared in Examples and Comparative Examples were evaluated according to the following contents.

### 1. Particle (Base Resin) Size

The prepared particles were dried, the dried particles were dispersed in ethanol at a level of about 10%, and then the particle size (D₅₀) was measured using a PSA device. At this time, D₅₀ means the particle size corresponding to 50% cumulative volume in the particle size distribution.

### 2. Spheroidization Rate (%) of Particles (Base Resin)

The spheroidization rate was determined by confirming the length (L) of the longest dimension among the prepared particles through SEM, and calculating the percentage ratio (D₅₀/L) of the particle size (D₅₀) measured in the item 1 to the length (L).

### 3. Crack Rate (%) of Particles (Base Resin)

The particle crack rate was determined by confirming the number of particles that have defects on the surface or are cracked without maintaining their spherical shape among the prepared particles through SEM, and calculating the percentage ratio of the number of particles maintaining a spherical shape relative to the total particles.

### 4. Shape of Particle (Base Resin) Pores

The pores formed on the surface and inside of the dried particles were observed Using a SEM (JEOL JSM7610F-Plus) device.

### 5. Pore Size of Particles (Base Resin) and Porosity of Particles

The pore size of the particles was measured using Mercury intrusion porosimetry (MIP) (Autopore V9600, micromeritics).

### 6. Surface Pore Size

20 or more pore sizes (wherein the pore size means the length of the largest dimension in the pore shape) were determined on a SEM image of the particle surface, and the arithmetic mean value was calculated. By the calculated surface pore size, the degree of openness of the particle surface (which cannot be determined by MIP) can be indirectly confirmed.

TABLE 5
Characteristics of particles (base resin) of Examples

| | | | | | Particle | | |
|---|---|---|---|---|---|---|---|
| | Particle | Particle | Particle | Pore | pore size | Porosity of | Average |
| | size | spheroidization | crack | shape | by MIP | particles | surface |
| | (D₅₀, μm) | rate (%) | rate (%) | image | (nm) | (%) | pore(nm) |
| Example 1 | 49.8 | >90 | <5 | See | 40.1 | 20.5 | 146 |
| | | | | FIG. 1 | | | |
| Example 2 | 51.6 | >90 | <5 | See | 38.4 | 20.3 | 159 |
| | | | | FIG. 2 | | | |
| Example 3 | 50.8 | >90 | <10 | See | 35.4 | 21.1 | 131 |
| | | | | FIG. 3 | | | |
| Example 4 | 48.8 | >90 | <10 | — | 35.1 | 20.8 | 123 |

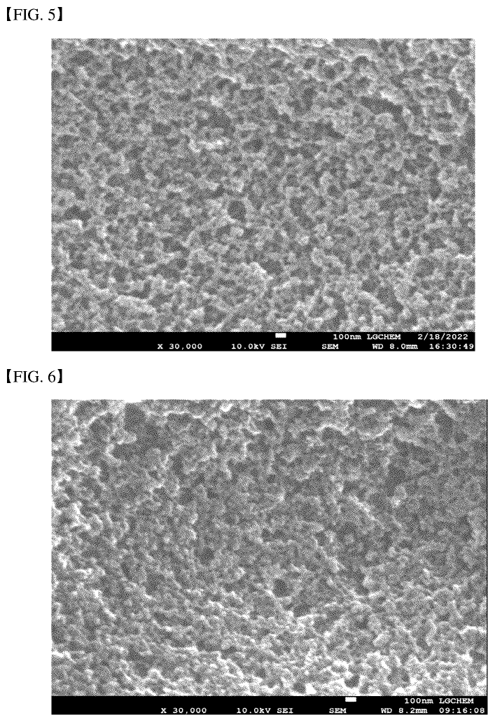

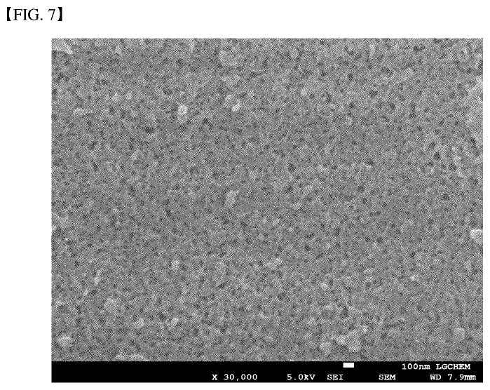

TABLE 6
Characteristics of particles (base resin) of Comparative Examples

| | | | | | Particle | | |
|---|---|---|---|---|---|---|---|
| | Particle | Particle | Particle | Pore | pore size | Porosity of | Average |
| | size | spheroidization | crack | shape | by MIP | particles | surface |
| | (D₅₀, μm) | rate (%) | rate(%) | image | (nm) | (%) | pore(nm) |
| Comparative | 61.45 | >90 | <20 | — | 17.8 | 17.2 | 81 |
| Example 1 | | | | | | | |
| Comparative | 41.9 | >90 | <5 | — | 24.6 | 18.0 | 100 |
| Example 2 | | | | | | | |
| Comparative | 44.6 | >90 | <5 | — | 27.7 | 20.2 | 105 |
| Example 3 | | | | | | | |
| Comparative | 46.5 | >90 | <5 | — | 21.8 | 21.4 | 89 |
| Example 4 | | | | | | | |
| Comparative | 54.1 | >90 | <5 | See | 11.1 | 12.4 | 82 |
| Example 5 | | | | FIG. 4 | | | |
| Comparative | 53.7 | >90 | <5 | See | 11.7 | 12.8 | 79 |
| Example 6 | | | | FIG. 5 | | | |
| Comparative | 49.2 | >90 | <5 | See | 10.8 | 13.9 | 62 |
| Example 7 | | | | FIG. 6 | | | |
| Comparative | — | — | >90 | — | — | — | — |
| Example 8 | | | | | | | |
| Comparative | — | — | >90 | — | — | — | — |
| Example 9 | | | | | | | |
| Comparative | 44.2 | >90 | <10 | — | 9.8 | 9.3 | 99 |
| Example 10 | | | | | | | |
| Comparative | 45.1 | >90 | <5 | See | 7.8 | 8.1 | 45 |
| Example 11 | | | | FIG. 7 | | | |
| Comparative | — | — | >90 | — | — | — | — |
| Example 12 | | | | | | | |

* In the case of Comparative Examples 8~9 and 12, the crack rate of the particles was high, and thus, evaluation regarding particle characteristics could not be conducted.

### Evaluation 2: Evaluation of Non-Specific Adsorption Rate

The particles prepared in Examples and Comparative Examples were processed according to the following contents, and the related contents were evaluated.

### 1. Epoxy Content, SBC and Non-Specific Adsorption Rate of Particles (Base Resin)

(1) Analysis of epoxy content (μmol/g) of particles (base resin)
1) 0.1 g of particles (DI water was used for a control) were mixed with 1 mL HCl/acetone solution (1/40 (v/v)) and sonicated for 4 minutes (UCP-02 from Jeiotech).
2) An indicator solution (0.1% cresol red and 0.1% thymol blue were mixed in a 1:3 volume ratio and adjusted to pH 7.0) was added to the mixed solution in the item 1).
3) Titration was performed with 0.1M NaOH until the color of the solution becomes purple-blue.
4) The epoxy content was calculated using the following Equation.

$$Epoxy content ( mmol ) = [ V 0 ( ml ) - V ( ml ) ] \times C NaOH ( mol / L ) \qquad [ Equation ]$$

in the above Equation,
V0: Amount of NaOH added to control sample using DI water instead of particles (mL)
V: Amount of NaOH added to the experimental group sample containing epoxy (mL)
CNaOH: NaOH concentration used for titration
5) The calculated epoxy content was divided by the weight of the particles (0.1 g was used in the item 1) beforehand), and the epoxy content per weight of particle (base resin) was confirmed.

(2) SBC (Static binding capacity, mg/mL) was measured (Reference: Ind. Eng. Chem. Res. 2013, 52, 26, 8800-8811, Biochemical Engineering Journal 145 (2019) 53-61)
1) 50 μL of a base resin slurry was prepared, and the antibody solution (IgG from human serum, 5 mg/mL PBS, pH 7.4) was thawed, and then 1×PBS was added to prepare the antibody solution at 3 mg/mL.
2) After mixing the resin slurry and the antibody solution, the IgG was bounded to the resin using a mixer at room temperature for 1 hour.
3) After precipitating the resin, the supernatant (or stored as needed) was removed, and washed by injecting 5 mL of 1× PBS.
4) After precipitating the resin, the washing solution (or stored as needed) was removed, and 500 μL of 100 mM glycine-HCl (pH 2.5) eluate was injected to elute the IgG.
5) After precipitating the resin, the eluate was collected and stored at 4° C.
6) The IgG concentration was measured in the eluate at 280 nm using UV-vis (8453 UV-visible spectrophotometer from Agilent). After IgG binding, the IgG content was measured in each of the supernatant, washing solution, and eluate, and the IgG content in each solution was calculated using the IgG calibration curve.

(3) Non-specific adsorption rate (%) when measuring SBC

Based on 100% of the total IgG content (weight) of the supernatant, washing liquid, and eluate confirmed in the item 2)-6) above, the content (%) of IgG confirmed in the washing liquid was substituted into the non-specific adsorption rate (%) when measuring SBC.

2. Epoxy Content (μMol/g), Hydrophilic Functional Group Content (μMol/g), And Non-Specific Adsorption Rate (%) after Epoxy Deactivation on Particles (Base Resin).

(1) Epoxy deactivation
1) The base resin was injected into 1-thioglycerol solution (0.5 M 1-thioglycerol +0.1 M sodium sulfate+0.1 M EDTA (pH8.5)), and allowed to react at 37° C. for 17 hours. For Comparative Example 1, ethanol amine was used instead of 1-thioglycerol.
2) The particles was precipitated using a method such as centrifugation, and then the supernatant was removed. Washed 3 times with DIW.
(2) Epoxy content after epoxy deactivation (μmol/g)

Epoxy deactivated particles, that is, particles surface-treated with 1-thioglycerol, were prepared by drying at room temperature, and the epoxy content of particles surface-treated with 1-thioglycerol was measured in the same manner as described above.

(3) Content of hydrophilic functional group (μmol/g)

The hydrophilic functional group content of the particles surface-treated with 1-thioglycerol was measured as shown in <Equation> below.

Hydrophilic functional group content after 1-thioglycerol surface treatment=(Epoxy content of particles before 1-thioglycerol surface treatment)×Number of hydroxyl functional groups in 1-thioglycerol <Equation>

### (4) Non-Specific Adsorption Rate (%)

The epoxy deactivated particles were dried. Then, the particles were mixed with a 10 mg/mL solution of BSA (sigma A9418) dissolved in DIW at a concentration of 31.3 mg/mL. Then, the mixed solution was stirred at room temperature for 18 hours, washed 5 times in DIW, and dried at room temperature. The non-specific adsorption rate was calculated through the ratio of the content of BSA adsorbed to the particles relative to the total BSA content added during stirring at room temperature. At this time, the content of BSA adsorbed on the particles was analyzed using Pierce™ BCA Protein Assay Kit (Thermo Fischer).

TABLE 7
Evaluation after epoxy deactivation of particles (base resin) of Examples

| | | | | Base resin treated by epoxy deactivation | | | |
|---|---|---|---|---|---|---|---|
| | | | | | | | Non-specific |
| | | | | | | | adsorption |
| | Base resin | | | | | Hydrophilic | rate under |
| | | | Non-specific | | | functional | condition |
| | | | adsorption | | Epoxy | group | of BSA |
| | | | rate during | | content | content | 10 mg/mL |
| | Epoxy | | SBC | Epoxy | after | after | after |
| | content | SBC | measurement | deactivation | treatment | treatment | treatment |
| | (μmol/g) | (mg/mL) | (%) | Agent | (μmol/g) | (μmol/g) | (%) |
| Example 1 | 1891 | 0.56 | 4.7 | 1-thioglycerol | <50 | 3682 | 0.068 |
| Example 2 | 1752 | 0.59 | 4.7 | 1-thioglycerol | <50 | 3504 | 0.43 |
| Example 3 | 1300 | 0.60 | 5.2 | 1-thioglycerol | <50 | 2600 | 0.71 |
| Example 4 | 1418 | 0.59 | 4.8 | 1-thioglycerol | <50 | 2836 | 0.66 |

TABLE 8
Evaluation after epoxy deactivation of particles (base resin) of Comparative Examples

| | | | | Base resin treated by epoxy deactivation | | | |
|---|---|---|---|---|---|---|---|
| | | | | | | | Non-specific |
| | | | | | | | adsorption |
| | Base resin | | | | | Hydrophilic | rate under |
| | | | Non-specific | | | functional | condition |
| | | | adsorption | | Epoxy | group | of BSA |
| | | | rate during | | content | content | 10 mg/mL |
| | Epoxy | | SBC | Epoxy | after | after | after |
| | content | SBC | measurement | deactivation | treatment | treatment | treatment |
| | (μmol/g) | (mg/mL) | (%) | Agent | (μmol/g) | (μmol/g) | (%) |
| Comparative | 695 | 0.9 | 6.2 | Ethanol | 597 | 98 | 4.4 |
| Example 1 | | | | amine | | | |
| Comparative | 762 | 0.73 | 7.8 | — | — | — | — |
| Example 2 | | | | | | | |
| Comparative | 554 | 1.49 | 6.2 | 1-thioglycerol | <50 | 1008 | 8.75 |
| Example 3 | | | | | | | |
| Comparative | 200 | — | — | 1-thioglycerol | <50 | 400 | 9.81 |
| Example 4 | | | | | | | |
| Comparative | 503 | — | — | 1-thioglycerol | <50 | 1006 | 6.98 |
| Example 5 | | | | | | | |
| Comparative | 680 | — | — | 1-thioglycerol | <50 | 1360 | 7.11 |
| Example 6 | | | | | | | |
| Comparative | 596 | — | — | 1-thioglycerol | <50 | 1192 | 7.29 |
| Example 7 | | | | | | | |
| Comparative | Impossible to | Same as | Same as | Same as | Same as | Same as | Same as |
| Example 8 | measure due | the left | the left | the left | the left | the left | the left |
| | to particle | | | | | | |
| | cracks | | | | | | |
| Comparative | Impossible to | Same as | Same as | Same as | Same as | Same as | Same as |
| Example 9 | measure due | the left | the left | the left | the left | the left | the left |
| | to particle | | | | | | |
| | cracks | | | | | | |
| Comparative | 987 | 0.69 | 7.1 | 1-thioglycerol | <50 | 1974 | 1.55 |
| Example 10 | | | | | | | |
| Comparative | 2081 | 0.61 | 5.5 | 1-thioglycerol | <50 | 4162 | 1.13 |
| Example 11 | | | | | | | |
| Comparative | Impossible to | Same as | Same as | Same as | Same as | Same as | Same as |
| Example 12 | measure due | the left | the left | the left | the left | the left | the left |
| | to particle | | | | | | |
| | cracks | | | | | | |

In the case of Comparative Example 2, since the non-specific adsorption rate of the base resin itself was confirmed as a high value, subsequent repetitive non-specific adsorption rate tests (i.e., related experiments after epoxy deactivation treatment) were not conducted.
In Comparative Example 4, which had a significantly low epoxy content in the base resin itself, a non-specific adsorption rate test for the base resin was not conducted. In the same sense, a non-specific adsorption rate test was not conducted on the base resin of Comparative Examples 5~7.

1. A method for preparing porous particles, the method comprising:
mixing a continuous phase composition and a dispersed phase composition to form a mixture, wherein the dispersed phase composition include an epoxy group-containing polymerizable monomer, a crosslinking agent, and a porogen, and
subjecting the mixture to a suspension polymerization reaction to prepare a suspension polymerization product, wherein the suspension polymerization product includes a unit derived from the polymerizable monomer and the crosslinking agent,
wherein the dispersed phase composition includes 50 parts by weight or more of the epoxy group-containing polymerizable monomer based on 100 parts by weight of the crosslinking agent, and
wherein the porogen includes a porogen having a difference in Hansen solubility parameters from the epoxy group-containing polymerizable monomer that is in a range of from 1.30 to 4.70.

2. The method for preparing porous particles according to claim 1, wherein:
the dispersed phase composition includes 150 parts by weight or more of the epoxy group-containing polymerizable monomer based on 100 parts by weight of the crosslinking agent.

3. The method for preparing porous particles according to claim 1, wherein:
the porogen includes a porogen having a difference in Hansen solubility parameters from the epoxy group-containing polymerizable monomer is that is in a range of from 2.50 to 4.50.

4. The method for preparing porous particles according to claim 1, wherein:
the porogen comprises an acetate-based porogen.

5. The method for preparing porous particles according to claim 1, wherein:
the dispersed phase composition comprises 45% by weight or more of the porogen, based on 100% by weight of the total content of the epoxy group-containing polymerizable monomer, the crosslinking agent and the porogen.

6. The method for preparing porous particles according to claim 1, wherein:
the crosslinking agent comprises a crosslinking agent that does not contain a hydroxyl group (—OH), or is a crosslinking agent that does not contain a hydroxyl group (—OH).

7. The method for preparing porous particles according to claim 1, wherein:
the continuous phase composition comprises water and a polymeric surfactant.

8. The method for preparing porous particles according to claim 1, wherein:
the suspension polymerization product has a particle shape, and
the particles have a D₅₀ size in the range of 30 to 70 μm.

9. The method for preparing porous particles according to claim 1, wherein:
the suspension polymerization product has a particle shape, and
the particles have a pore size in the range of from 30 to 500 nm, as measured by the mercury intrusion method, and a particle average surface pore size in the range of 110 to 240 nm, by an arithmetic average of the sizes of 20 or more pores observed on the particle surface.

10. The method for preparing porous particles according to claim 1, wherein:
the suspension polymerization product
is in the form of particles having a porosity in the range of 15 to 40%.

11. The method for preparing porous particles according to claim 1,
further comprising making the surface of the suspension polymerization product hydrophilic through deactivation of the epoxy groups.

12. The method for preparing porous particles according to claim 11, wherein:
the suspension polymerization product has an epoxy group derived from the epoxy group-containing polymerizable monomer, and
an epoxy deactivator having an epoxy reactive group and a hydrophilic group different therefrom is reacted with the suspension polymerization product having an epoxy group to make the surface of the suspension polymerization product hydrophilic.

13. The method for preparing porous particles according to claim 12, wherein:
the epoxy deactivator comprises at least one of a thiol group and an amine group as the epoxy reactive group, and comprises a hydroxy group as a hydrophilic group different from the epoxy group.

14. Porous particles comprising a polymerization unit derived from a polymerizable monomer having an epoxy group and a crosslinking agent,
wherein the particles satisfy the following features:
a particle size (D₅₀) in the range of 30 to 70 μm,
a pore size in the range of 30 to 500 nm, as measured by the mercury intrusion method,
a particle average surface pore size in the range of 110 to 240 nm, by an arithmetic average of the sizes of 20 or more pores observed on the particle surface, and
a porosity in the range of 15 to 40%.

15. The porous particles according to claim 14, wherein:
the porous particles are surface-treated with an epoxy deactivator having an epoxy reactive group and a hydrophilic group different from the epoxy reactive group.
